(19) United States
(12) Patent Application Publication
(10) Pub. No.: US 2003/0077822 A1
(43) Pub. Date: Apr. 24, 2003

(54) Enzyme solution for culturing primate embryonic stem cells and method of culturing using it

(76) Inventors: Norio Nakatsuji, Kyoto-shi (JP); Hirofumi Suemori, Kyoto-shi (JP)

Correspondence Address: Edward D. Grieff, Esq., Hale and Dorr LLP, 1455 Pennsylvania Avenue, NW, Washington, DC, 20004, US

(21) Appl. No.: 10/244,748

(22) Filed: Sep. 17, 2002

(30) Foreign Application Priority Data
Oct. 16, 2001 (JP) 2001-317694

(51) Int. Cl. C12N 5/08; C12N 5/06

(52) U.S. Cl. 435/366; 435/364

(57) ABSTRACT

An enzyme solution for subculturing primate embryonic stem (ES) cells and a method of culturing primate ES cells is described herein. The enzyme solution comprises trypsin, calcium chloride and a serum substitute. The culturing method comprises the step of culturing primate ES cells in a solution comprising trypsin and calcium chloride, and optionally a serum substitute. The solution and method of this invention enable one to stably maintain and propagate ES cells derived from a primate, such as monkey or human, for a long period in an undifferentiated state and with a normal karyotype.

### RELATED APPLICATION

This application claims priority to Japanese Application No. 2001-317694 filed Oct. 16, 2001, the disclosure of which is incorporated by reference in its entirety.

### FIELD OF THE INVENTION

The present invention relates to an enzyme solution for subculturing primate embryonic stem (ES) cell lines, and a method of subculturing the ES cells using the solution.

### BACKGROUND OF THE INVENTION

ES cells are undifferentiated pluripotent cells that are derived from early stage embryos and propagate rapidly. They exhibit a nature similar to embryonal carcinoma cells, have a high in vitro differentiation potency, and differentiate into various kinds of cells merely by culturing their aggregate mass. ES cells have been established from pre-implantation stage embryos, and they have pluripotency to differentiate into various cell types derived from the three germ layers: the ectoderm, mesoderm and endoderm (M. J. Evans and M. H. Kaufman, Nature 292: 154-156 (1981); G. R. Martin, Proc. Natl. Acad. Sci. USA. 78: 7634-7638 (1981)). There is a great desire in the art to isolate and propagate ES cells from, in particular, a primate including human because ES cells can supply any type of cell and tissue which can be utilized for transplantation in a mammal and other applications including human, drug discovery, and gene therapy.

Generally, ES cell lines are usually established by culturing cells derived from the inner cell mass (ICM) of blastocysts. Alternatively, dissociated cells of morula or blastocysts, implantation of which is delayed, may be used. These embryonic cells immediately differentiate into epithelium-like cells and such. Therefore, in order to maintain these cells in their undifferentiated state, they must be subcultured on a feeder cell layer prepared from mouse embryonic fibroblasts or STO cells, the culture medium of which needs to be frequently changed so as to retain a suitable cell density. Standard methods used for establishing ES cells are described in Evans et al., Nature 292: 154-156 (1981), Martin et al., Proc. Natl. Acad. Sci. USA 78: 7634-7638 (1981), E. J. Robertson ed. “Embryo-derived stem cells” (IRL Press Ltd., Oxford (1987) “Teratocarcinoma and Embryonic Stem Cells, A Practical Approach” and so on.

A prior art method for establishing the ES cell lines using fibroblasts as a feeder cell layer is performed, for example, as follows. First, early stage embryos, in particular, blastocysts or blastocysts implantation of which is delayed, are cultured and transplanted onto a feeder cell layer. Expansion growth of trophoblasts in the periphery of embryos is initiated; ICM present in the interior of early stage embryos propagate in a dome form on expanded trophoblasts. When ICM is sufficiently propagated, it is separated and dispersed to subculture on a fresh feeder cell layer. Out of subcultured ICM-derived cells, a very small number of cells will continue to propagate while maintaining an undifferentiated state. These undifferentiated cells are further subcultured and propagated to establish the ES cell lines.

A known culture medium is prepared by supplementing the DME culture medium, which is used as a basal culture medium, with a mixture of unessential amino acids, wherein a mixture of nucleic acids, mercaptoethanol, newborn bovine serum and/or fetal bovine serum are added thereto (Doetschman T. C. et al., J. Embryol. Exp. Morph. 87: 27-45 (1985)). It has been reported that, for establishment and maintenance of mouse ES cell lines, addition of a given amount of EC cell culture supernatant (Martin G R., Proc. Natl. Acad. Sci. USA 78: 7634-7638 (1981)), or the buffalo rat liver cell culture supernatant (BRL-CM) to the above-mentioned culture medium, inhibited differentiation and promoted propagation at the same time (Smith, A. G & Hooper, M. L., Dev. Biol. 121: 1-9 (1987)). The activity contained in these media was called differentiation-inhibiting activity (DIA). Thereafter, DIA was found to be due to one kind of cytokine called leukemia-inhibiting factor (LIF) (Williams, R. L. et al., Nature 336: 684-687 (1988)). ES cells have an apparently indefinite lifespan under conditions that maintain their undifferentiated state, in the presence of a feeder cell layer and/or leukemia-inhibiting factor (LIF) (R. Williams et al, Nature 336: 684-687 (1988)).

Human ES cell lines were also established and displayed a similar differentiation potency to mouse ES cells (J. A. Thomson et al., Science 282: 1145-1147 (1998); J. A. Thomson et al., Dev. Biol. 38: 133-165 (1998); B. E. Reubinoff et al., Nat. Biotechnol. 18: 399-404 (2000)). However, mouse ES cells are different from human ES cells in many aspects. Therefore, it is difficult to stably maintain and propagate ES cells from a primate, such as monkey and human, for a long period in the enzyme solution or the medium which has been used for culturing mouse ES cell lines.

### SUMMARY OF THE INVENTION

For mouse ES cells, a stable culturing and propagation method has been already established. In contrast, further improvement in methods for culturing ES cells from a primate, including human, is still needed. In particular, a cell propagation method that maintains stem cells in their undifferentiated state and inhibits spontaneous cell differentiation is needed, as is a dissociation method with a small cell loss when the cells are subcultured by transferring the cells to an increased number of culture vessels. The present invention addresses the above problems and provides an optimal enzyme solution for subculturing ES cells derived from a primate, including human, and an effective method for culturing and propagating primate ES cells.

The present inventors established four ES cell lines from blastocysts from cynomolgus monkey (Macaca fascicularis). By using an improved trypsin solution and a serum-free medium having a specified composition, cells were well maintained in an undifferentiated state and with a normal karyotype even after 6 months or more of culturing. Since ES cells from cynomolgus monkey have the closest natures to human ES cells, it is considered that a trypsin solution for subculturing cynomolgus monkey and a culture medium, can be used to stably maintain and propagate ES cells derived from other primates, including human. The present invention specifically includes the following:

(1) an enzyme solution for culturing primate embryonic stem (ES) cells, wherein said solution comprises trypsin, calcium chloride and a serum substitute;

(2) the enzyme solution according to (1), which comprises trypsin in a concentration ranging from 0.05 to 0.5 w/v % and calcium chloride in a concentration ranging from 0.5 to 5 mM;

(3) the enzyme solution according to (1), wherein said solution consists essentially of trypsin, calcium chloride and a serum substitute;

(4) a method of culturing primate ES cells, the method comprising the steps of:

(a) culturing an inner cell mass on a feeder cell layer;

(b) dissociating cells contained within said inner cell mass; and

(c) subculturing the dissociated cells on a feeder cell layer in a solution comprising trypsin and calcium chloride;

(5) the method of (4), wherein said solution further comprises a serum substitute;

(6) the method of (4), wherein said solution contains trypsin in a concentration ranging from 0.05 to 0.5 w/v % and calcium chloride in a concentration ranging from 0.5 to 5 mM; and

(7) the method of (5), wherein said solution consists essentially of trypsin, calcium chloride and a serum substitute.

### DETAILED DESCRIPTION OF THE INVENTION

The present invention relates to an enzyme solution for subculturing primate ES cells. The enzyme solution contains trypsin, calcium chloride, and a serum substitute. In the solution, trypsin is contained in an amount ranging from about 0.05 to about 0.5%, and preferably from about 0.1 to about 0.3%. Herein, “%” means “w/v %” unless otherwise specified. Calcium chloride is contained in an amount ranging from about 0.5 to about 5 mM, and preferably from about 1 to about 2 mM.

The serum substitute suitable for ES cell that is to be cultured may be selected from many known serum substitutes. It should both suppress differentiation of primate ES cells and allow their growth. Examples of the serum substitute include Knockout Serum Replacement (KNOCKOUT™ SR, Invitrogen, hereinafter abbreviated as KSR), Serum Replacement 1 (Sigma-Aldrich), Serum Replacement 2 (Sigma-Aldrich), Fetal Clone I (Hyclone), and Fetal Clone II (Hyclone). KSR is preferably used. Although the concentration of the serum substitute in the present enzyme solution varies depending on the kind of the serum substitute and so on, a person skilled in the art would routinely determine the appropriate concentration. It is contained in an amount ranging from about 10 to about 30%, and preferably from about 15 to about 25%.

These components are dissolved in buffered saline, such as PBS, Hank's balanced salt solution, Earle's balanced salt solution, and Tyrode's salt solution, preferably PBS.

The enzyme solution according to the present invention is suitable for establishing ES cells from a primate including cynomolgus monkey and human. It can also be used for dissociating the cells by pipetting or other such means.

Further, the present invention relates to a method of culturing primate ES cells. Specifically, as shown in Examples but not limited thereto, for example, blastocysts from a primate, such as monkey or human, are prepared, and the ICM isolated therefrom is plated on a feeder cell layer. Cells used for the feeder layer are mouse embryonic fibroblasts, STO cell line, and so on, and preferably mouse embryonic fibroblasts. The ICM is then cultured for 3 to 7 days in a culture medium. Any known culture medium for ICM can be used. Examples thereof include a 1:1 mixture of Ham's nutrient mixture F-12 and Dulbecco's modified Eagle's medium (DMEM) supplemented with 0.1 mM 2-mercaptoethanol and 15% fetal bovine serum (FBS), DMEM supplemented with 10% FBS and 10% newborn calf serum (Evans, M. J., Kaufman, M. H., “Establishment in culture of pluripotent stem cells from mouse embryo.”Nature, 292, 154-156 (1981)), and DMEM supplemented with 10% newborn calf serum (Martin, G R., “Isolation of a pluripotent cell line from early mouse embryos cultured in medium conditioned by teratocarcinoma stem cells.” Proc. Natl. Acad. Sci. USA. 78, 7634-7638 (1981)). Serum can be replaced with a serum substitute as exemplified above, such as KSR. The culture medium is adjusted to 7.0 to 8.0.

Before subculturing, the cells can be dissociated by pipetting using a capillary glass or other means. The dissociation can be performed in a solution containing trypsin and EDTA, a solution containing trypsin and calcium chloride, or an enzyme solution of the present invention.

The dissociated cells are then plated on a feeder layer and cultured at 35 to 40° C. in the culture medium. The culture medium is changed every day.

In the solution containing trypsin and calcium chloride, which can be used in cell dissociation and subculturing, trypsin is contained in an amount ranging from about 0.05 to about 0.5%, and preferably from about 0.1 to about 0.3%, and calcium chloride is contained in an amount ranging from about 0.5 to about 5 mM, and preferably from about 1 to about 2 mM.

The solution can be combined with a serum-free medium to increase the efficiency of cloning of primate ES cells. It becomes possible to maintain ES cells in an undifferentiated state for a long period without performing periodic collection of stem cell colonies using a medium containing fetal bovine serum (FBS), as is required in the prior art method. As the serum-free medium, for example, a medium containing about 15 to 25%, and preferably about 20% KSR is contemplated.

The enzyme solution and culturing method of the present invention is suitable for subculturing primate ES cells. Exemplary primate include but are not limited to monkeys, such as cynomolgus monkey, Rhesus monkey and common marmoset, and humans. The present invention may have similar effects on ES cells from an animal other than primates, such as rabbit, rat, cow, and pig, cell cultures of which have also been difficult to establish and subculture In addition, the present invention may also be used for genetically altered ES cells.

The present invention enables the stable maintenance and growth of ES cells derived from a primate, including human, while maintaining the ES cells in an undifferentiated state and with a normal karyotype.

The present invention is described in greater detail by way of Examples but is not to be construed as being limited thereto.

### EXAMPLE 1

Establishment of Cynomolgus ES Cell Lines

Blastocysts from the cynomolgus monkey were prepared by in vitro culturing for 7 to 10 days following in vitro fertilization (IVF) or intracytoplasmic sperm injection (ICSI) (R Torii et al., Primat ES 41: 39-47 (2000); Hosoi et al., in press). The ICM was separated by immunosurgery using rabbit antiserum against cynomolgus spleen cells (D. Solter and B. KnowlES, Proc. Natl. Acad. Sci. USA. 72: 5099-5102 (1973)). The separated ICM was plated on a feeder cell layer of mitomycin C-inactivated mouse embryonic fibroblasts. The cells were cultured in a 1:1 mixture of Ham's nutrient mixture F-12 (Sigma) and DMEM supplemented with 0.1 mM 2-mercaptoethanol, 1000 unit/ml ESGRO™ (Gibco) and 15% FBS (JRH). The cells were also cultured in the same medium, with the exception that FBS was replaced with 20% KSR (Gibco). After 3 to 7 days, before transferring onto a fresh feeder cell layer, the expanded ICM was dissociated by pipetting with a fine capillary glass in 0.25% trypsin/1 mM EDTA. The colonies having the stem cell-like morphology were recovered, dissociated mechanically or using trypsin/EDTA, and transferred to a feeder cell layer for expansion. Subsequent subculturing of ES cell lines was carried out using 0.25% trypsin in PBS containing 20% KSR and 1 mM CaCl₂ or 1 mg/ml type IV collagenase (Gibco) in DMEM. After these processes, seven independent stem cell lines were isolated. However, three lines of them were lost during subsequent expansion. Four cell lines (designated as CMK 5, 6, 7 and 9) were successfully propagated, and used in further analysis.

The present inventors established and characterized four ES cell lines using 32 blastocysts derived from Macaca fascicularis. The efficiency of this establishment is comparable to the efficiency for human ES cell lines (5 from 14 blastocysts) (Thomson et al., (1998), supra). These cell lines had similar morphology to the human and other monkey ES cell lines. Cynomolgus ES cells formed tightly packed but flatter colonies than mouse ES cells. Each cell had a high nucleus/cytoplasm ratio and prominent nucleoli. They did not form domed colonies, which are typical for mouse ES cells. They showed an epithelium-like appearance immediately after subculturing, but formed tightly packed colonies within a few days of growth. The cynomolgus ES cells have similar morphology to other primate ES cells, which have been previously reported, and formed flatter colonies as compared with the domed colonies that are typical for mouse ES cells. As reported for other primate ES cells, LIF was not effective for maintaining the cynomolgus ES cells in an undifferentiated state. When the ES cells were plated on gelatinized dishes without the feeder cell layer, they differentiated and ceased growth even in the presence of LIF. Thus, the feeder cell layer is indispensable for maintaining these ES cell lines. Partial populations of the ES cells spontaneously differentiated even in the presence of the feeder cell layer and LIF. In some cases, it was necessary to manually collect the stem cell-like colonies to maintain the ES cell lines.

### EXAMPLE 2

Improvement of Culture Medium and Subculturing Method

As previously reported for other ES cells, cynomolgus ES cells exhibited poor cloning efficiency. This may cause a problem when isolating cell clones after gene transfection, or when selecting a transformant. Although a conditioned medium for cynomolgus ES cells had no influence on the plating efficiency, it was considered that an unknown paracrine factor produced by ES cells, or contact between cells may be necessary for the growth of primitive ES cells. Since ES cells spontaneously differentiated during subculturing, limited dissociation was necessary in order to maintain stem cell clusters of 10 to 50 cells to enable continued growth. Thus, the standard dissociation procedure for mouse ES cells using trypsin caused excessive damage to the monkey ES cells. Without trypsin, however, the cells were not properly dissociated. After testing various conditions, the present inventors found an adequate method for efficient subculturing by using 0.25% trypsin supplemented with 1 mM CaCl₂ and 20% KSR (Gibco).

The autonomous appearance of differentiated cells during ES cells culturing was remarkably decreased when fetal bovine serum (FBS) was replaced with KSR in the culture medium. There is a possibility that FBS contains a differentiation-inducing factor, such as a growth factor, that is not present in KSR. In the serum-free medium, cynomolgus ES cells were maintained in an undifferentiated state for a long period without periodic collection of stem cell colonies, which had been required when using the FBS medium. In the KSR medium, however, ES cells exhibited a flatter morphology and slower growth rates than in the FBS medium. Even so, splitting of the ES cell culture occurred every 3 to 4 days. It is reported that bFGF enhances the cloning efficiency of human ES cells (M. Amit et al., Dev. Biol. 227, 271-278 (2000)). However, the addition of bFGF was not effective in increasing the cloning efficiency or maintaining the cynomolgus ES cells in the undifferentiated state.

### EXAMPLE 3

Determination of Karyotype

After 3 to 5 months of culturing of ES cells according to the method described in Example 1, karyotype was determined by the conventional G band method. The results are shown in Table 1.

TABLE 1
Karyotype analysis

| Cell line | Passage (month) | Normal/counted (%) | Sex |
|---|---|---|---|
| CMK5 | 3(1) | 17/20(85) | Male |
| CMK6 | 47(6) | 14/20(70) | Male |
| CMK7 | 4(1) | 15/20(75) | Female |
| CMK9 | 18(3) | 16/20(80) | Female |

The number of chromosomes of each cell line was counted in various passage numbers. Approximate culture periods are shown in months. Spreads showing diploid (40+XX, or XY) chromosomes were counted as normal. Karyotypic sex is also shown.

As shown in Table 1, cynomolgus ES cells retained a normal karyotype even after 3 to 6 month culturing or even after recovery from the freezing and thawing process. Two cell lines were male karyotype and two cell lines were female karyotype. These ES cells retained the normal karyotype even after long term culturing.

What is claimed is:

1. An enzyme solution for culturing primate embryonic stem cells, wherein said solution comprises trypsin, calcium chloride and a serum substitute.

2. The enzyme solution according to claim 1, which comprises trypsin in a concentration ranging from 0.05 to 0.5 w/v % and calcium chloride in a concentration ranging from 0.5 to 5 mM.

3. The enzyme solution according to claim 1, wherein said solution consists essentially of trypsin, calcium chloride and a serum substitute.

4. A method of culturing primate embryonic stem cells, the method comprising the steps of:
(a) culturing an inner cell mass on a feeder cell layer;
(b) dissociating cells contained within said inner cell mass; and
(c) subculturing the dissociated cells on a feeder cell layer in a solution comprising trypsin and calcium chloride.

5. The method of claim 4, wherein said solution further comprises a serum substitute.

6. The method of claim 4, wherein said solution contains trypsin in a concentration ranging from 0.05 to 0.5 w/v % and calcium chloride in a concentration ranging from 0.5 to 5 mM.

7. The method of claim 5, wherein said solution consists essentially of trypsin, calcium chloride and a serum substitute.
